(19) United States
(12) Patent Application Publication
(10) Pub. No.: US 2003/0216626 A1
(43) Pub. Date: Nov. 20, 2003

(54) Fluorescence judging method and apparatus

(75) Inventors: Kazuhiro Tsujita, Kaisei-machi (JP); Eiji Ogawa, Kaisei-machi (JP)

Correspondence Address: SUGHRUE MION, PLLC, 2100 Pennsylvania Avenue, NW, Washington, DC, 20037-3213, US

(73) Assignee: FUJI PHOTO FILM CO., LTD.

(21) Appl. No.: 10/401,146

(22) Filed: Mar. 28, 2003

(30) Foreign Application Priority Data
Mar. 28, 2002 (JP) 092283/2002
Mar. 28, 2002 (JP) 092284/2202

(51) Int. Cl. A61B 5/00

(52) U.S. Cl. 600/321

(57) ABSTRACT

A CCD imaging element obtains a narrow band image and a wide band image from fluorescence emitted from a subject upon the irradiation by an excitation light. A reflectance image is obtained of light reflected from the subject upon the irradiation by a near-infrared light. A fluorescence value computing means obtains a normalized fluorescence computed value by dividing the value of each pixel of the narrow band image by that of the corresponding pixel of the wide band image, and obtains a computed fluorescence yield rate by dividing each pixel value of the wide band image by the corresponding pixel value of the reflectance image. A judging portion judges the tissue state of the subject based on the two-dimensional distribution point of the two aforementioned computed values and prerecorded computed value distribution data of normalized fluorescence computed values and computed fluorescence yield rates for tissues having known tissue states.

### BACKGROUND OF THE INVENTION

1. Field of the Invention

The present invention relates to a fluorescence judging method and apparatus for judging the tissue state of a target subject portion on the basis of the fluorescence light emitted from the target subject upon the irradiation thereof with an excitation light.

2. Description of the Related Art

There have been proposed fluorescence judging apparatuses that project an excitation light of a predetermined wavelength range onto a target subject portion such as a living tissue or the like and analyze the fluorescence data of the fluorescence emitted from the target subject to determine the tissue state of the target subject. Regarding these types of fluorescence judging apparatuses, there are apparatuses that determine the tissue state based on the analysis of the fluorescence emitted from a diagnostic fluorescent dye that has been absorbed by the target subject in advance, and apparatuses that determine the tissue state on the basis of autofluorescence, that is, without the use of fluorescent dyes. In many cases, these types of fluorescence judging apparatuses are provided built in to an endoscope or a colposcope for insertion into a body cavity, or a surgical microscope or the like.

Early versions of autofluorescence judging apparatuses, such as that shown in FIG. 11, determined whether a tissue was in a normal or a diseased state by taking advantage of the fact that the intensity of fluorescence emitted from diseased tissue is smaller than the intensity of fluorescence emitted from normal tissue. However, because there is unevenness on the surface of a tissue portion, the distance between the excitation light source and the target subject is not uniform, resulting in an uneven intensity of excitation light on the surface of the target subject. Meanwhile, the intensity of the fluorescence emitted from a tissue in the normal state is substantially proportional to that of the excitation light, and the intensity of the excitation light decreases in inverse proportion to the square of the aforementioned distance. Therefore, there are cases in which fluorescence of a stronger intensity is detected from a diseased tissue that is closer to the light receiving means than a normal tissue located further away, resulting in an erroneous result when the judgment of the tissue state is determined solely on the basis of the fluorescence intensity.

To prevent errant judgments of this type, systems have been proposed for determining the tissue state of a target subject by obtaining a computed fluorescence value based on the ratio of the intensity of the excitation light detected at the target tissue portion and the intensity of the fluorescence emitted from the target subject upon the irradiation thereof by the aforementioned excitation light, that is, a value corresponding to the fluorescence yield rate, which is a value unaffected by the distance between the excitation light and the target subject or the angle of incidence and the like.

However, when obtaining the above-described value corresponding to the fluorescence yield rate, because the excitation light, which is formed light having wavelengths in the UV (ultra-violet) range to the visible spectrum, is absorbed by various substances of living tissue, even if the intensity distribution of the reflected excitation light is measured, an accurate measurement of the intensity distribution of the excitation light absorbed by the living tissue can not be obtained. Therefore, systems for judging the tissue state of a target subject have been proposed employing a strategy of obtaining a value corresponding to the fluorescence yield rate by projecting a near infrared light, which exhibits more consistent absorption characteristics compared to light having a wavelength in the UV to visible spectrum, onto the living tissue as a reference light, and using the intensity of the reflected near infrared light instead of the intensity of the reflected excitation light to obtain the computed fluorescence yield rate by dividing the intensity of the fluorescence by the intensity of the reflected near infrared light, and basing the evaluation of the tissue state on the thus obtained fluoresce yield rate value. That is to say, by obtaining the above-described computed value of the fluorescence yield rate, factors determining the intensity of the fluorescence yield which are dependent on the on the distance between the excitation light source and the fluorescence receiving portion and the target subject can be cancelled, whereby the judgment of the tissue state can be performed based on a computed value corresponding to only the difference of the fluorescence yield rate.

On the other hand, the development of fluorescence judging apparatuses that utilize the fact that the spectral form of the fluorescence emitted from a tissue in a normal state and the spectral form of the fluorescence emitted from a tissue in a diseased state are different, as shown in FIG. 11, is progressing. For example, apparatuses have been proposed for judging the tissue state based on a comparison of the intensity of the fluorescence in the green wavelength range and the intensity of the fluorescence in the red wavelength range (e.g. U.S. Pat. Nos. 5,507,287 and 5,769,792). Further, apparatuses have also been proposed for judging the tissue state of a target subject by comparing the spectral form of the fluorescence emitted from a tissue known to be in the normal state, which has been obtained in advance, and the spectral form of the fluorescence emitted from the target subject (e.g. U.S. Pat. No. 5,579,773).

Further, there has been proposed an apparatus that utilizes a normalized fluorescence computed value, wherein the intensity of the narrow band fluorescence obtained from a target subject has been standardized by the intensity of the wide band fluorescence, to judge the tissue state of the target subject (e.g. Japanese Unexamined Patent Publication No.10(1998)-225436). According to the aforementioned apparatus: a narrow band fluorescence image of the wavelength band near 480 nm, at which the difference between the intensity of the fluorescence emitted from a diseased portion and that emitted from a healthy portion is most pronounced, and a wide band fluorescence image of the wavelength band in the 430-730 nm range are obtained; a normalized fluorescence computed value is obtained by dividing the pixel values of the narrow band fluorescence image by the pixel values of the wide band fluorescence image; the tissue state is judged for each pixel on the basis of the normalized fluorescence computed value; and a pseudo color image is displayed based on the tissue state determined for each pixel. That is to say, by obtaining the above-described normalized fluorescence computed value, the factors of the fluorescence intensity dependent on the distance between the target subject and the excitation light source as well as the fluorescence receiving portion is cancelled, whereby the tissue state can be judged based upon the computed value corresponding to only the difference in the spectral form of the fluorescence.

However, upon evaluation of the results of in vivo measurements, if the tissue state is judged based on the one parameter obtained from the fluorescence as described above, it has been discovered that there are cases for which it is difficult to obtain a sufficient degree of accuracy. Attention has been focused on improving the judgment accuracy by using a combination of a plurality of parameters in judging the tissue state, and there has been proposed an apparatus that judges the tissue state based on a plurality of parameters obtained from fluorescence (e.g. U.S. Pat. No. 6,516,217). According to the aforementioned apparatus, the fluorescence intensity or the fluorescence yield rate is combined with the normalized fluorescence computed value to judge the tissue state, whereby an improvement in the judgment accuracy with respect to discriminating between a tissue in a diseased state and a tissue in normal state has been demonstrated.

However, according to the apparatus described in U.S. Pat. No. 6,516,217, the intensity of the fluorescence emitted from the target subject, the computed fluorescence yield rate, or the normalized fluorescence computed value are each compared to a respective, predetermined threshold value to judge whether the tissue is in a diseased or a normal state, and on the basis of the logical product of each of these judgments, a final judgment of the tissue state is performed. However, there are cases in which the tissue state will be judged to be in a normal state in one of the preliminary judgments and to be in a diseased state in another, wherefore it is difficult to say that the judgment obtained in such cases is accurate.

Further, there are many cases in which a large amount of fluorescence emitting mucous fluid, digestive fluids, saliva, foam, waste material and the like is attached to a target subject. When excitation light is projected onto a target subject that has a large amount of any of the aforementioned fluorescence emitting matter adhered thereto (hereafter referred to as an unclean tissue), fluorescence is emitted from the mucous fluid, digestive fluids, saliva, foam, waste material and the like. The fluorescence emitted from an unclean tissue oftentimes differs in intensity and spectral form compared to that emitted from a clean tissue. According to conventional tissue state judging apparatuses, the reading for this type of unclean tissue has often been categorized as “Difficult to verify normal tissue”, or “Difficult to verify diseased tissue”. However, because the conventional tissue state judging apparatuses are unable to judge that a target subject is an unclean tissue, it becomes impossible to discriminate whether a reading such as “Difficult to verify normal tissue”, or “Difficult to verify diseased tissue” has been obtained for a target subject due to the fact that the target subject is an unclean tissue to which a large quantity of fluorescence emitting mucous fluid, digestive fluids, saliva, foam, waste material and the like is adhered to, or the fact that it is a clean tissue and the “Difficult to verify normal tissue”, or “Difficult to verify diseased tissue” reading has been obtained because that is the type of tissue the target subject is, giving rise to a problem in that the reliability of the tissue state judgment result is lowered.

### SUMMARY OF THE INVENTION

The present invention has been developed in consideration of the forgoing problems, and it is an object of the present invention to provide a tissue state judgment method and apparatus for judging, based on the fluorescence emitted from a target subject upon the irradiation thereof with an excitation light, the tissue state of the target subject, and which is capable of improving the accuracy of the judgment.

It is a further object of the present invention to provide a tissue state judgment method and apparatus capable of improving the reliability of the tissue state judgment result.

The fluorescence judging method according to the present invention comprises the steps of:

recording in advance a two-dimensional distribution of the fluorescence emitted from a plurality of tissues, each of which the respective tissue state is known, upon the irradiation thereof by an excitation light, wherein the two-dimensional distribution is formed of a normalized fluorescence computed value corresponding to the spectral form of the fluorescence emitted from each tissue of which the tissue state is known, and a computed fluorescence yield rate corresponding to the fluorescence yield rate of the fluorescence, and a computed value distribution data formed based on the relation of the two-dimensional distribution to the tissue state of each of the tissues of which the tissue state is known,

detecting the fluorescence data of the fluorescence emitted from a target subject upon the irradiation thereof with an excitation light,

obtaining the normalized fluorescence computed value and the computed fluorescence yield rate based on the detected fluorescence data, and

judging the tissue state of the target subject on the basis of both of the computed values and the prerecorded computed value distribution data.

The fluorescence judging apparatus according to the present invention comprises:

a memory means for recording in advance a two-dimensional distribution of the fluorescence emitted from a plurality of tissues, each of which the respective tissue state is known, upon the irradiation thereof by an excitation light, wherein the two-dimensional distribution is formed of a normalized fluorescence computed value corresponding to the spectral form of the fluorescence emitted from each tissue of which the tissue state is known, and a fluorescence yield rate computed value corresponding to the fluorescence yield rate of the fluorescence, and a computed value distribution data formed based on the relation of the two-dimensional distribution to the tissue state of each of the tissues of which the tissue state is known,

an excitation light emitting means for projecting an excitation light onto the target subject,

a fluorescence detecting means for detecting the fluorescence data of the fluorescence emitted from the target subject upon the irradiation thereof with the excitation light,

a computing means for obtaining the normalized fluorescence computed value and the fluorescence yield rate computed value based on the detected fluorescence data, and

a judging means for judging the tissue state of the target subject based on both of the computed values and the prerecorded computed value distribution data.

Note that “detecting the fluorescence data of the fluorescence emitted from the target subject” can refer to, for example, the fluorescence of a predetermined wavelength range that has been obtained as an image by use of a CCD imaging element or the like, or a point of fluorescence that has been obtained by a point measurement process employing a single optical fiber.

Further, “normalized fluorescence computed value” refers to a computed value that reflects the spectral form of the fluorescence and which is a computed value corresponding to the intensity rate between the fluorescence of different wavelength bands obtained from the target subject. The different wavelength bands can be, for example, a narrow band near the 480 nm wavelength and a narrow band near the 630 nm wavelength.

Still further, the normalized fluorescence computed value can be a value obtained by dividing the intensity of the fluorescence of a narrow wavelength range (e.g. 430-530 nm wavelength band) by the intensity of the fluorescence of a wide wavelength range (e.g. the entire bandwidth, or the 430-730 nm wavelength range).

The term “fluorescence yield rate” refers to the ratio of the intensity of the excitation light projected onto the target subject to the intensity of the fluorescence emitted from the target subject upon the irradiation thereof by said excitation light. Further, the referents of “computed fluorescence yield rate” can include, for example, a computed value obtained by projecting a reference light onto the target subject and using the intensity of the reflected light of the reference light instead of the intensity of the excitation light, and dividing the intensity of the fluorescence emitted from the target subject by the intensity of the reference light reflected from the target subject. Regarding the reference light, a near infrared light exhibiting comparatively uniform reflectance properties for a wide range of tissue types can be used. In addition, though a slight reduction in accuracy is incurred, a normal illumination light can be used as the reference light. Note that if it is possible to maintain a low level of fluctuation in the distance between the excitation light emitting portion, e.g., the distal end of the scope portion of an endoscope, and the target subject, the fluorescence intensity can be used as the computed fluorescence yield rate.

Further, for cases in which the two-dimensional distribution point of the normalized fluorescence computed value and the computed fluorescence yield rate of the fluorescence emitted from the target subject is not included in the computed value distribution data, the judging means can be a means that judges that the target subject is an unclean tissue.

Note that “two-dimensional distribution point of the normalized fluorescence computed value and the computed fluorescence yield rate” refers to, for example, the point in a two-dimensional space wherein the normalized fluorescence computed value is the y axis and the computed fluorescence yield is the x axis, which is plotted based on the normalized fluorescence computed value and the computed fluorescence yield rate obtained of the fluorescence emitted from the target subject. Further, the “cases in which the two-dimensional distribution point . . . is not included in the computed value distribution data” refers to, more specifically, cases in which the two-dimensional point does not fall within the range of values of the two-dimensional space formed based on the normalized fluorescence computed values and computed fluorescence yield rates that have been obtained of the tissues having a known tissue state.

In addition, the fluorescence judging apparatus can further comprise a display means for simultaneously displaying the computed value distribution data and the judgment result.

Another fluorescence judging method according to the present invention for judging the tissue state of a target subject based on the fluorescence emitted from the target subject upon the irradiation thereof with an excitation light comprises the steps of:

recording in advance the distribution data of a plurality of characteristic quantities obtained based on the fluorescence data of the fluorescence emitted from a clean tissue upon the irradiation thereof by an excitation light,

detecting the fluorescence data of the fluorescence emitted from the target subject upon the irradiation thereof by the excitation light,

obtaining, based on the fluorescence data detected by the detecting means, the plurality of characteristic quantities, and

judging, based on the plurality of characteristic quantities and the distribution data, that the target subject is an unclean tissue.

Another fluorescence judging apparatus according to the present invention for judging the tissue state of a target subject based on the fluorescence emitted from the target subject upon the irradiation thereof with an excitation light comprises:

a memory means for recording in advance the distribution data of a plurality of characteristic quantities obtained based on the fluorescence data of the fluorescence emitted from a clean tissue upon the irradiation thereof by an excitation light,

a detecting means for detecting the fluorescence data of the fluorescence emitted from the target subject upon the irradiation thereof by the excitation light,

a characteristic quantity obtaining means for obtaining, based on the fluorescence data detected by the detecting means, the plurality of characteristic quantities, and

a judging means for judging, based on the plurality of characteristic quantities and the distribution data, that the target subject is an unclean tissue.

Note that here, “clean tissue” refers to a tissue to which a large quantity of fluorescence emitting mucous fluid, digestive fluids, saliva, foam, waste material and the like is not adhered. Further, “unclean tissue” refers to a tissue to which a large quantity of fluorescence emitting mucous fluid, digestive fluids, saliva, foam, waste material and the like is adhered. The referents of “a plurality of characteristic quantities” can include, more specifically, the fluorescence intensity, the spectral form of the fluorescence, a normalized fluorescence computed value corresponding to the spectral form of the fluorescence and a computed fluorescence yield rate corresponding to the fluorescence yield rate of the fluorescence, and the like. The plurality of characteristic quantities can also be the normalized fluorescence computed value corresponding to the spectral form of the fluorescence and a computed fluorescence yield rate corresponding to the fluorescence yield rate of the fluorescence.

Further, for cases in which the clean tissue is composed of a plurality of tissues of which the state is known, if the memory means is a means for recording in advance the distribution data of a plurality of characteristic quantities for each of the tissues of which the state is known, the judging means can be a means for judging the tissue state of the target subject based on the plurality of characteristic quantities obtained by the characteristic quantities obtaining means and the distribution data of the characteristic quantities for each of the tissues of which the state is known.

Still further, the aforementioned other fluorescence judging apparatus can also be a fluorescence detecting apparatus wherein:

the excitation light emitting means projects excitation light onto an observation area,

the detecting means detects as an image the fluorescence data of the fluorescence emitted from the observation area,

the characteristic quantities obtaining means obtains, based on the fluorescence data, a plurality of characteristic quantities for each pixel of the image, and

the judging means judges, based on the plurality of characteristic quantities and the distribution data that has been prerecorded in the memory means, for each pixel that the target subject corresponding to the pixel is an unclean tissue, further comprising

a fluorescence diagnostic image forming means for forming a fluorescence diagnostic image based on the judgment result obtained by the judging means, and

a display means for displaying the fluorescence diagnostic image.

The fluorescence diagnostic image forming means can be a means for subjecting a pixel unit, which is formed of a predetermined number of pixels, to a display correction process for cases in which the ratio of the number of pixels of a pixel unit that are judged to represent unclean tissue is greater than or equal to a predetermined value.

Note that “display correction process” refers to a process capable of differentiating the pixel units for which the ratio of the number of pixels thereof that have been judged to represent an image of unclean tissue is above a predetermined value from the other pixel units, and can consist of, for example, displaying a pixel unit targeted for the display correction process in a special color, or with no color, etc.

Further, the fluorescence diagnostic image forming means can be a means for appending, corresponding to the ratio of the number of pixels of a pixel unit, which is formed of a predetermined number of pixels, judged to be an image of unclean tissue, reliability data to the pixel unit.

Still further, each of the above-described fluorescence judging apparatuses can be provided as part of a fluorescence endoscope apparatus having an endoscope portion for insertion into a body cavity.

The inventors of the present invention continued to research judgment methods for judging tissue state by use of parameters obtained of fluorescence after the filing the application for the invention disclosed in U.S. Pat. No. 6,516,217. As a result, the inventors of the present invention discovered that there is a close correlation, as shown in FIG. 1, between the two-dimensional distribution formed by the computed fluorescence yield rate and the normalized fluorescence computed value, and the tissue state.

FIG. 1 is a graph charting the computed value distribution data relating the computed fluorescence yield rate and the normalized fluorescence computed value obtained of the fluorescence emitted from each of a plurality of normal tissues, precancerous tissues, and diseased (cancerous) tissues, and the respective tissue state. It is evident from FIG. 1 that the computed fluorescence yield rate and the normalized fluorescence computed value for each type of tissue state are concentrated within a predetermined distribution region.

That is to say, according to the fluorescence judging method and apparatus according to the present invention, because the tissue state of a target subject is judged based on the computed fluorescence yield rate and the normalized fluorescence computed value obtained of the fluorescence emitted from the target subject, and a prerecorded computed value distribution data, the accuracy of the judgment is improved.

If the normalized fluorescence computed value is obtained by dividing the fluorescence intensity of a narrow wavelength range by the fluorescence intensity of a wide wavelength range, the possibility that a division by 0 will be performed can be reduced, and a normalized fluorescence computed value appropriately corresponding to the spectral form of the fluorescence can be obtained.

If the judging means is a means for judging that the target subject is an unclean tissue for cases in which the two-dimensional distribution of the normalized fluorescence computed value and the computed fluorescence yield rate of the fluorescence emitted from the target subject is not included in the computed value distribution data, by displaying this type of judgment result on a monitor or the like, it becomes possible to improve the reliability of the diagnosis obtained by a diagnostician diagnosing the tissue state.

Further, if the fluorescence judging apparatus further comprises a display means for simultaneously displaying the computed value distribution data and the judgment result obtained by the judging means, the diagnostician can view both the computed value distribution data and the judgment result obtained by the judging means on the same image, whereby the utility of the fluorescence judging apparatus is improved.

Still further, the inventors of the present invention have further discovered, in their research of judgment methods and apparatuses for judging the tissue state of a target subject based on a plurality of parameters of the fluorescence emitted from a target subject upon the irradiation thereof with an excitation light, that the distribution region of the two-dimensional distribution of a plurality of characteristic quantities (e.g. the computed fluorescence yield rate and the normalized fluorescence computed value) obtained of the fluorescence emitted from a clean tissue and that of the two-dimensional distribution of a plurality of characteristic quantities obtained of the fluorescence emitted from an unclean tissue are different.

FIG. 2 is a graph charting the two-dimensional distribution data relating the computed fluorescence yield rate and the normalized fluorescence computed value, obtained of the fluorescence emitted from clean tissues and unclean tissues, to which mucous, digestive fluids, saliva, foam, and waste material or the like is adhered, of a plurality of tissues in the normal, precancerous, and diseased (cancerous) states, to each tissue. It is evident from FIG. 2 that the distribution region of the computed fluorescence yield rate and the normalized fluorescence computed value obtained of the fluorescence emitted from a clean tissue and that of the computed fluorescence yield rate and the normalized fluorescence computed value obtained of the fluorescence emitted from an unclean tissue are different.

Accordingly, if the computed fluorescence yield rate and the normalized fluorescence computed value obtained of the fluorescence emitted from the target subject do not correspond to the computed fluorescence yield rate and the normalized fluorescence computed value emitted from a clean tissue, the target subject can be judged to be an unclean tissue.

That is to say, according to the above-described other fluorescence judging method and apparatus of the present invention, by prerecording the distribution data of a plurality of characteristic quantities obtained based on the fluorescence data of the fluorescence emitted from a clean tissue upon the irradiation thereof by an excitation light, as shown for example in FIG. 2, then obtaining a plurality of characteristic quantities based on the fluorescence data of the fluorescence emitted from a target subject upon the irradiation thereof by an excitation light, because it becomes possible to judge that the tissue state of the target subject is an unclean tissue on the basis of the plurality of characteristic quantities of the target subject and the distribution data, the reliability of the judgment result can be improved.

If the computed fluorescence yield rate corresponding to the fluorescence yield rate of the fluorescence and the normalized fluorescence computed value corresponding to the spectral form of the fluorescence are used as the plurality of characteristic quantities, because it can be judged that the target subject is an unclean tissue on the basis of the fluorescence yield rate and the spectral form of the fluorescence, the judgment of the tissue state can be accurately performed.

If the normalized fluorescence computed value is obtained by dividing the fluorescence intensity of a narrow wavelength range by the fluorescence intensity of a wide wavelength range, the possibility that a division by 0 will be performed can be reduced, and a normalized fluorescence computed value appropriately corresponding to the spectral form of the fluorescence can be obtained.

Further, for cases in which the clean tissue is composed of a plurality of tissues of which the state is known, if the memory means is a means for recording in advance the distribution data of a plurality of characteristic quantities for each of the tissues of which the state is known, the judging means can be a means for judging the tissue state of the target subject based on the plurality of characteristic quantities obtained by the characteristic quantities obtaining means and the distribution data of the characteristic quantities for each of the tissues of which the state is known; whereby it becomes possible to simultaneously perform the operation to judge whether or not the target subject is an unclean tissue, and to judge the tissue state of a clean tissue.

Still further, if the excitation light emitting means projects excitation light onto an observation area, and the fluorescence judging apparatus detects as an image the fluorescence data of the fluorescence emitted from the observation area, obtains, based on the fluorescence data, a plurality of characteristic quantities for each pixel of the image, and judges, based on the plurality of characteristic quantities and the distribution data that has been prerecorded in the memory means, for each pixel that the target subject corresponding to the pixel is an unclean tissue, and further comprises a fluorescence diagnostic image forming means for forming a fluorescence diagnostic image based on the judgment result obtained by the judging means and a display means for displaying the fluorescence diagnostic image, the judgment result can be displayed as an image, whereby the convenience and utility of the fluorescence judging apparatus can be improved.

Even further, if the fluorescence diagnostic image forming means is a means for subjecting a pixel unit, which is formed of a predetermined number of pixels, to a display correction process for cases in which the ratio of the number of pixels of a pixel unit that are judged to represent unclean tissue is greater than or equal to a predetermined value, the pixel units of which the aforementioned is greater than or equal to the predetermined value can be displayed in a color or the like enabling differentiation thereof from the other pixel units, whereby it becomes possible for the diagnostician to easily discriminate the image regions in which there are a large number of unclean tissues.

In addition, if the fluorescence diagnostic image forming means is a means for appending, corresponding to the ratio of the number of pixels of a pixel unit, which is formed of a predetermined number of pixels, judged to be an image of unclean tissue, reliability data to the pixel unit, the degree of reliability of the pixel units can be displayed.

### BRIEF DESCRIPTION OF THE DRAWINGS

FIG. 1 is an explanatory graph illustrating characteristic quantities,

FIG. 2 is an explanatory graph illustrating characteristic quantities,

FIG. 3 is a schematic drawing of a fluorescence endoscope apparatus according to the first embodiment of the present invention,

FIG. 4 is a schematic drawing of a mosaic filter,

FIG. 5 is a schematic drawing of a switching filter,

FIG. 6 illustrates the tissue state judging method,

FIG. 7 illustrates the display screen,

FIG. 8 is a schematic drawing of a fluorescence endoscope apparatus according to the second embodiment of the present invention,

FIG. 9 is a schematic drawing of a fluorescence endoscope apparatus according to the third embodiment of the present invention,

FIG. 10 illustrates the display screen, and

FIG. 11 is a graph illustrating the fluorescence intensity spectrum of the fluorescence emitted from a normal tissue and the fluorescence intensity spectrum of the fluorescence emitted from a diseased tissue.

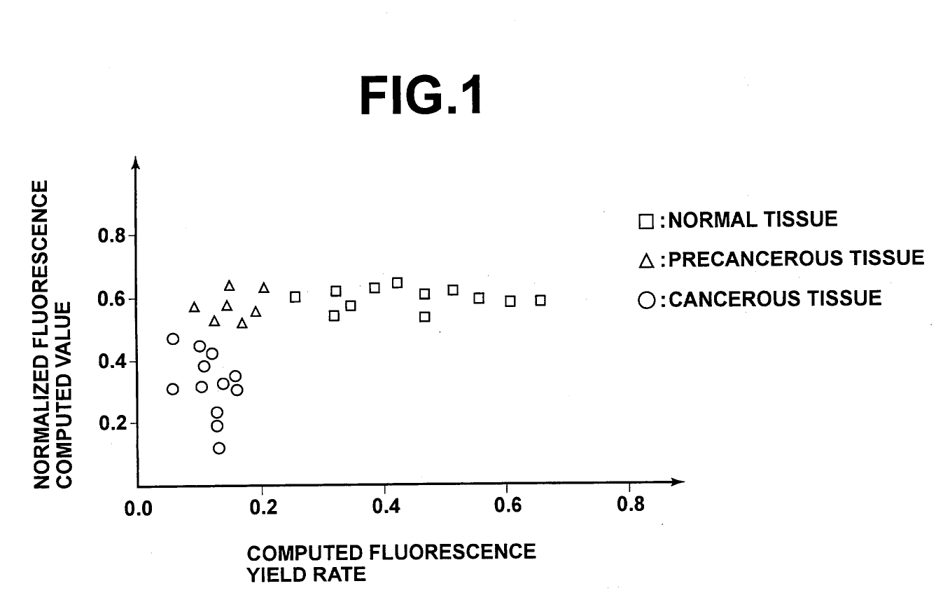

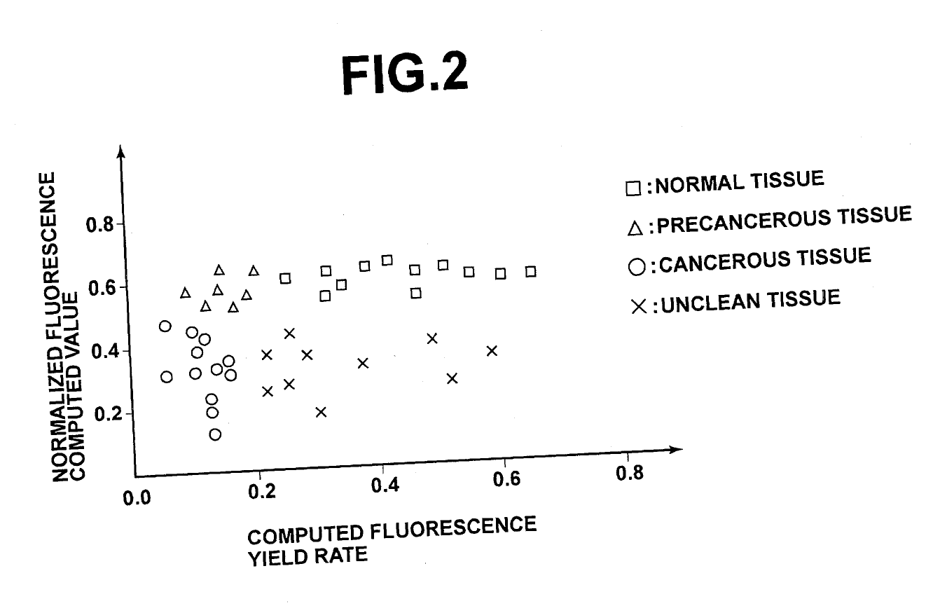

### DESCRIPTION OF THE PREFERRED EMBODIMENTS

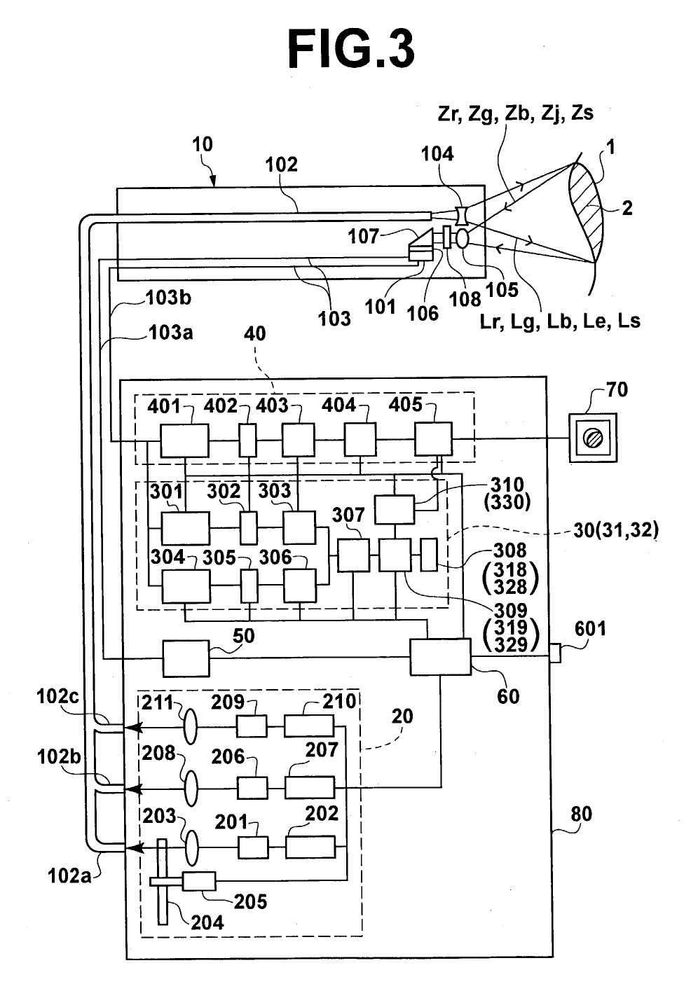

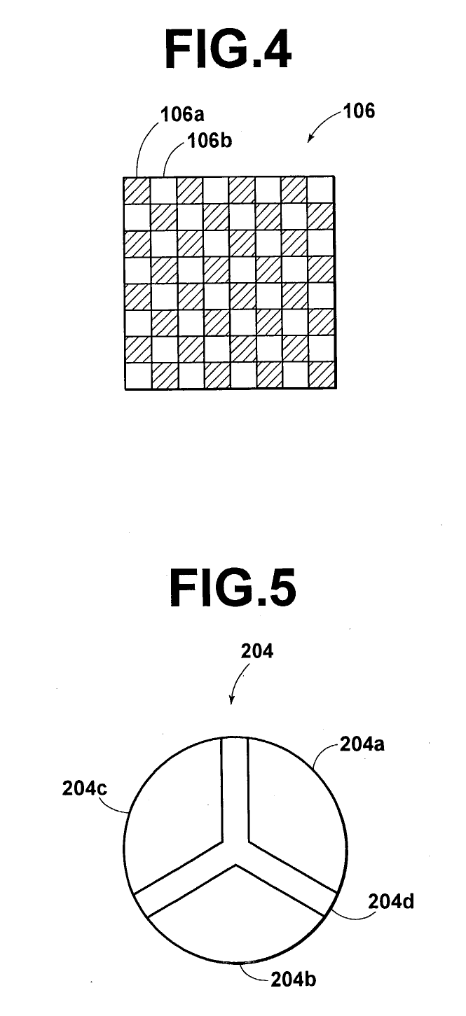

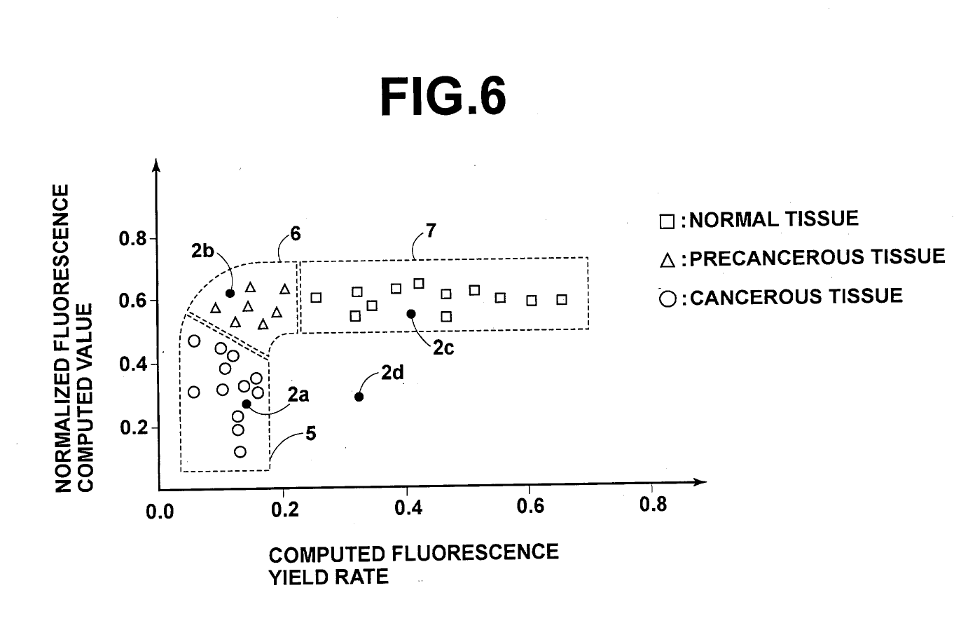

Hereinafter the preferred embodiments of the present invention will be explained with reference to the attached drawings. First, with reference to FIGS. 3-6, a fluorescence endoscope apparatus implementing the fluorescence judging method and apparatus according to the first embodiment of the present invention will be explained. FIG. 3 is a schematic drawing of the fluorescence endoscope apparatus; FIGS. 4 and 5 are schematic drawings of a mosaic filter, and a switching filter, respectively, loaded into the fluorescence endoscope apparatus; and FIG. 6 is a graph illustrating computed value distribution data.

The fluorescence endoscope apparatus according to the first embodiment of the present invention operates a standard image mode for displaying a standard image which is a normal color image, and a fluorescence diagnostic image mode for displaying a fluorescence diagnostic image represented by a pseudo color image of the tissue state judgment result for each target subject 2 of an observation area 1 on the basis of the fluorescence emitted from the observation area 1 upon the irradiation thereof by an excitation light. Switching between the two modes is performed by input operations from an input apparatus 601. Note that the region within the observation area 1 corresponding to each pixel of the CCD is a target subject 2, and the tissue state judgment is performed for each target subject 2. That is to say, a plurality (equivalent to the number of pixels of the CCD) of target subjects 2 collectively forms the observation area 1.

According to the standard image mode, the reflected light of R (red) light Lr, G (green) light Lg and B (blue) light Lb sequentially projected onto the surface of the observation area 1 is obtained by a CCD imaging element 101, and the formed standard image is displayed on a monitor 70 by standard color signal processing.

According to the fluorescence diagnostic image mode: a narrow band fluorescence image and a wide band fluorescence image are obtained, by use of the CCD imaging element 101, of the fluorescence emitted from an observation area 1 upon the irradiation thereof by an excitation light Le; an IR reflectance image is obtained, by use of the CCD imaging element 101, from the imaged IR reflectance Zs formed of the light reflected from the observation area 1 upon the irradiation thereof by a near infrared light; a normalized fluorescence computed value is obtained by dividing the pixel values of the narrow band fluorescence image by the pixel values of the wide band fluorescence image; a computed fluorescence yield rate is obtained by dividing the pixel values of the wide band fluorescence image by the pixel values of the IR reflectance image; the tissue state of each target subject 2 of the observation area 1 is judged using the two computed values and the computed value distribution data that has been prerecorded in the memory means 308; and the judgment results are displayed on the monitor 70 as a pseudo color image. Note that the computed value distribution data is explained in detail below.

As shown in FIG. 3, the fluorescence endoscope apparatus according to the first embodiment of the present invention comprises: a scope portion 10 which is provided with a CCD imaging element 101 at the distal end thereof, for insertion into the primary nidus and suspected diseased areas in a body cavity of a patient; an illumination unit 20 provided with a light source for emitting R (red) light Lr, G (green) light Lg and B (blue) light Lb used in obtaining standard images, an excitation light source for emitting an excitation light Le for obtaining fluorescence images, and a light source for emitting a near infrared light Ls as a reference light for obtaining IR reflectance images; a fluorescence image processing unit 30 for obtaining computed fluorescence values from the pixel values of the narrow band fluorescence image and wide band fluorescence image, and forming a fluorescence diagnostic image signal based on said computed fluorescence value; a standard image processing unit 50 for forming a standard image signal, and converting the standard image signal and the fluorescence diagnostic image signal outputted from the fluorescence image processing unit 30 to video signals; a CCD drive unit 6 for controlling the movement of the CCD 101; a controller 60 connected to each unit; an input apparatus 601 connected to said controller 60; and a monitor 70 that serves as a display means for displaying standard or fluorescence diagnostic images. Note that the illumination unit 20, the fluorescence image processing unit 30, the standard image processing unit 40, the CCD drive unit 50 and the controller 60 together form a processor portion 80. Connectors (not shown) enable the scope portion 10 and the processor portion 80, and the processor portion 80 and the monitor 70 to be connected so as to be freely separated.

The scope portion 10 is provided with a light guide 102 and a CCD cable 103 extending internally to the distal end thereof. An illuminating lens 104 and an objective lens 105 are provided at the distal end of the scope portion 10, further forward than the distal end of the light guide 102 and the CCD cable 103. A CCD imaging element 101 provided with an on-chip mosaic filter 106 formed of a plurality of microscopic region filters combined to form a mosaic pattern is provided at the distal end of the CCD cable 103, and a prism 107 is attached to said CCD imaging element 101. Further, an excitation light cutoff filter 108 for cutting off light having a wavelength less than or equal to 420 nm is provided between the objective lens 104 and the prism 107.

The CCD imaging element 101 is a frame transfer type imaging element provided with a receiving portion for converting the imaged fluorescence to an electrical signal, and an accumulation portion for temporarily storing and transferring the signal charge. The light guide 102 is formed as an integrated cable in which a light guide 102a for sequential light, a light guide 102b for excitation light and a light guide 102c for reference light are bundled, and each of said light guides is connected to the illuminating unit 20.

A drive line 103a for sending the drive signal of the CCD imaging element 101 and an output line 103b that reads out the image signal from the CCD imaging element 101 are combined in the CCD cable 103. The CCD drive unit 50 is connected to one end of the drive line 103a. One end of the output line 103b is connected to the fluorescence image processing unit 30 and the standard image processing unit 40.

As shown in FIG. 4, the mosaic filter 106 is formed of a plurality of narrow band pass filters 106a that transmit light having a wavelength in the 430-530 nm wavelength range and a plurality of full spectrum filters 106b that transmit all wavelengths of light, which are alternately arranged in a mosaic pattern. Each of the band pass filters 106a and 106b are in a one-to-one correspondence with a pixel of the CCD imaging element 101.

The illumination unit 20 comprises: a white light source 201 that emits a white light; a white light power source 202 electrically connected to said white light source 201; a switching filter 204 for sequentially switching between R light Lr, G light Lg and B light Lb; a filter rotating means 205 for rotating the switching filter 204; a GaN type semiconductor laser 206 that emits light having a wavelength of 410 nm for obtaining fluorescence images; a semiconductor laser power source 207 electrically connected to said GaN type semiconductor laser 206; a reference light source 209 that emits a reference light Ls, which is a near infrared light, for obtaining IR reflectance images; and a reference light power source 210 electrically connected to said reference light source 209.

As shown in FIG. 5, the switching filter 204 is formed of a R filter 204a that transmits R light Lr, a G filter 204b that transmits G light Lg, a B filter 204c that transmits B light Lb, and a mask portion 204d having a light shielding function. The mask portion 204d serves to transmit the signal charge from the light receiving portion of the CCD imaging element 101 to the accumulation portion when the sequential light (R light Lr, G light Lg and B light Lb) is not being emitted.

The fluorescence image processing unit 30 comprises: a signal processing circuit 101 for processing the image signal obtained at the CCD imaging element 101 when the excitation light Le has been emitted; an AD conversion circuit 302 for digitizing the image signal outputted from said signal processing circuit 301; an image memory 303 for storing the digitized image signal in respectively different memory regions for the narrow band fluorescence image, which corresponds to the image signal detected at the pixels of the CCD imaging element corresponding to the narrow band filters 106a of the mosaic filter, and the wide band fluorescence image, which corresponds to the image signal detected at the pixels of the CCD imaging element corresponding to the full spectrum filters 106b of the mosaic filter 106; a signal processing circuit 304 for processing the component of the image signal detected at the CCD imaging element 101 when the reference light Ls is emitted corresponding to the image signal detected at the pixels of the CCD imaging element 101 corresponding to the full spectrum filters 106b of the mosaic filter 106; an AD conversion circuit 305 for digitizing the image signal outputted from said signal processing circuit 304; an image memory 306 for storing the IR reflectance image formed by the digitized image signal; a computed fluorescence value obtaining portion 307 for dividing the pixel values of the narrow band fluorescence image stored in the image memory 303 by the pixel values of the wide band fluorescence image stored in the image memory 303, which have been obtained of respective adjacent pixels, to obtain a normalized fluorescence computed value, and dividing the pixel values of the wide band fluorescence image stored in the image memory 303 by the pixel values of the IR reflectance image stored in the image memory 306 to obtain a computed fluorescence yield rate; a recording portion 308 for recording a computed value distribution data such as that shown FIG. 6, a judging portion 309 for judging, by use of the normalized fluorescence computed value and the computed fluorescence yield rate obtained by the fluorescence computed value obtaining means 307 and the computed value distribution value recorded in the memory means 308, the tissue state for each pixel; and a fluorescence diagnostic image forming means 310 for assigning a color to each pixel on the basis of the judgment result to form a fluorescence diagnostic image and outputting the formed fluorescence diagnostic image signal to a video signal processing circuit 405, which is described below.

Here, the method of forming the computed value distribution data shown in FIG. 6 is explained. First, the computed fluorescence yield rate and the normalized fluorescence computed value are obtained according to the above-described method using the fluorescence endoscope apparatus of the current embodiment on the basis of the fluorescence emitted from clean tissues, to which a large quantity of fluorescence emitting mucous, digestive fluids, saliva, foam, and waste material or the like is not adhered, of which the tissue state has been determined in advance by another means to be a normal tissue state, a precancerous tissue state, or a diseased (cancerous) tissue state to form a two-dimensional distribution graph relating the obtained computed values to the normal, precancerous, and cancerous tissues. Next, the areas on the graph are delimited as an area 5 related to the cancerous tissue, an area 6 related to the precancerous tissue, and an area 7 related to the normal tissue. Each of the area 5 related to the cancerous tissue, an area 6 related to the precancerous tissue, and an area 7 related to the normal tissue are recorded in the memory means 308 as a computed value distribution data.

The standard image processing unit 40 comprises: a signal processing circuit 401 for processing the image signal detected by the pixels corresponding to the full-spectrum filters 106b of the mosaic filter 106 when the R light Lr, G light LG and B light Lb are emitted; an AD conversion circuit 402 for digitizing the signal outputted from the signal processing circuit 401; an image memory 403 for storing an image for each color (a R image, a G image, and a B image) the digitized image signal; a standard image forming portion 404 for forming a standard image from the images for each color stored in said image memory 403; and a video signal processing portion 405 for converting the standard image signal outputted from the standard image signal processing means 401 to a video signal and outputting said video signal when a standard image is to be displayed, and converting the fluorescence diagnostic image signal outputted from the fluorescence diagnostic image processing circuit 301 to a video signal and outputting said video signal when a fluorescence diagnostic image is to be displayed. The CCD drive unit 50 is a means for outputting operation control signals that control the operation timing of the CCD imaging element 101. The controller 60 is connected to each unit, and controls the operation timing.

Next, the operation of the fluorescence endoscope apparatus of the current embodiment of the present invention will be explained. According to the standard image mode, the sequential light is emitted, and the standard image is obtained and displayed. According to the fluorescence diagnostic image mode, the excitation light Le or the reference light Ls is emitted, a fluorescence image and an IR reflectance image are obtained in a time division manner, and a fluorescence diagnostic image is displayed.

First, the operation of the standard image mode will be explained. Before the image is obtained, the doctor inserts the scope portion 10 into a body cavity of the patient and positions the distal end of the scope portion 10 within close proximity of the observation area 1.

The explanation will proceed starting with the operation for obtaining the R image. The white light power source 202 is activated based on a signal from the controller 60, and white light is emitted from the white light source 201. The white light is focused by a focusing lens 203, and transmitted by the switching filter 204. In the switching filter 204, the R filter 204a is disposed in the light path based on a signal from the controller 60. Therefore, the white light becomes R light when transmitted by the switching filter 204. The R light enters the light guide, is guided to the distal end of the scope portion 10, and then projected onto the observation area 1 by the illuminating lens 104.

The R light Lr reflected from the observation area 1 is focused by the focusing lens 105, reflected by the prism 107, and focused on the CCD imaging element 101 as a R light reflectance image Zr. From the image signal outputted from the CCD imaging element 101, only the component thereof corresponding to that detected by the pixels corresponding to the full-spectrum filters 106b of the mosaic filter 106 is processed by the signal processing circuit 401 of the standard image processing unit 40, and outputted as a processed R image signal; the other component of the image signal is discarded. The R image signal is digitized by the AD conversion circuit 402, and stored in the R image memory region of the image memory 403. Then, the G image and B image are obtained according the same procedure and stored in the respective G image and B image memory regions of the image memory 403.

When the R image, G image, and B images have been stored in the image memory 403, the standard image forming means 404 forms, in synchronization with the display timing, a standard image signal from the three color images. The standard image signal is then converted to a video signal by the video signal processing circuit 405 and outputted to the monitor 70 to display the standard image, which is a color image.

Next, the operation of the fluorescence diagnostic image mode is explained. The doctor selects the fluorescence diagnostic image mode using the input apparatus 601. First, the excitation light power source 207 is activated, based on a signal from the controller 60, and the excitation light Le having a wavelength of 410 nm is emitted from the GaN type semiconductor laser 206. The excitation light Le is transmitted by a lens 208, enters the light guide 102b, is guided to the distal end of the scope portion 10, and then projected onto the observation area 1 from the illuminating lens 104.

The fluorescence emitted from the observation area 1 upon the irradiation thereof by the excitation light Le is focused by the focusing lens 105, reflected by the prism 107, transmitted by the mosaic filter 106, and focused on the CCD imaging element 101 as a fluorescence image Zj. Because the reflected light of the excitation light Le is cutoff by a cutoff filter, said reflected light does not enter the CCD imaging element 101. The fluorescence image Zj detected by the CCD imaging element 101 is photoelectrically converted to form an image signal corresponding to the intensity of the fluorescence, which is then outputted.

The signal outputted from the CCD imaging element 101 is processed by the signal processing circuit 301 of the fluorescence image processing unit 30, digitized by the AD conversion circuit 302, separated into a narrow band fluorescence image, which is formed of the image signal component that has been transmitted by the narrow band filters 106a of the mosaic filter, and a wide band fluorescence image, which is formed of the image signal component that has been transmitted by the full-spectrum filters 106b of the mosaic filter 106, which are then stored in a respective narrow band image memory region and a wide band image memory region of the image memory 303.

Next, the operation for obtaining the reference light Ls IR reflectance image Zr will be explained. The reference light power source 210 is activated, based on a signal from the controller 60, and the reference light, which is a near infrared light Ls, is emitted from the reference light source. The reference light Ls is transmitted by a lens 211, enters the light guide 102c, is guided to the distal end of the scope portion 10, and then projected onto the observation area 1 from the illuminating lens 104. The reflected light of the reference light LS reflected from the observation area 1 is focused by the focusing lens 105, reflected by the prism 107, transmitted by the mosaic filter 106, and focused on the CCD imaging element 101 as an IR reflectance image Zs. The IR reflectance image Zs detected by the CCD imaging element 101 is photoelectrically converted to form an image signal corresponding to the intensity of the light, which is then outputted.

From the image signal outputted from the CCD imaging element 101, only the component thereof corresponding to that detected by the pixels corresponding to the full-spectrum filters 106b of the mosaic filter 106 is processed by the signal processing circuit 304 of the fluorescence image processing unit 30, digitized by the AD conversion circuit 305, and stored as an IR reflectance image in the image memory 306.

When the IR reflectance image is stored in the image memory 306, the fluorescence computed value obtaining portion 307 divides the pixel values of the narrow band fluorescence image by the pixel values of the corresponding adjacent pixels of the wide band fluorescence image to obtain a normalized fluorescence computed value. Further, the fluorescence computed value obtaining portion 307 divides the pixel values of the wide band fluorescence image stored in the image memory 303 by the corresponding pixel values of the IR reflectance image stored in the memory 306 to obtain a computed fluorescence yield rate.

The judging portion 309 judges the tissue state of each target subject 2 on the basis of the two-dimensional distribution point of the normalized fluorescence computed value and the computed fluorescence yield rate thereof. As shown in FIG. 6a, because the two-dimensional distribution point 2a falls within the normal region 7 of the computed value distribution data stored in the memory means 308, the judging means 309 judges that the pixel corresponding to the target subject 2 represents a normal tissue. Further, because distribution point 2b falls within the precancerous region 6 of the computed value distribution data, the judging portion 309 judges the pixel corresponding to the target subject 2 represents a precancerous tissue; because the distribution point 2c falls within the diseased cancerous region 5, the judging portion 309 judges that the pixel corresponding to the target subject 2 represents a diseased (cancerous) tissue. Still further, because the distribution point 2d does not fall into any of the normal, precancerous and cancerous regions described above, the pixel corresponding to the target subject 2 is judged to represent an unclean tissue. Note that an unclean tissue is a tissue to which a large amount of fluorescence emitting mucous, digestive fluids, saliva, foam, or waste material is adhered; the tissue state cannot be judged based on the florescence emitted from an unclean tissue.

Based on the obtained judgment results, the fluorescence diagnostic image forming portion 310 forms a fluorescence diagnostic image signal by first assigning green for the pixels that have been judged to represent normal tissue, yellow to pixels that have been judged to represent precancerous tissues, red to pixels that have been judged to represent cancerous tissues and no color to the pixels judged to represent unclean tissue, and outputs the formed fluorescence diagnostic image signal to the video signal processing circuit 405. The video signal processing circuit 405 converts the fluorescence diagnostic image signal to a video signal and outputs the signal to the monitor 70 to display the fluorescence diagnostic image.

As made clear in the above explanation, according to the fluorescence endoscope apparatus of the current embodiment, because the tissue state of each target subject 2 of the observation area 1 is judged based on the two-dimensional distribution point formed by the normalized fluorescence computed value and the computed fluorescence yield rate of each said target subject and the computed value distribution data that has been prerecorded in the memory means 308, the accuracy of the tissue state judgment result can be improved. Therefore, a fluorescence diagnostic image more accurately corresponding to the tissue state of each target subject 2 of the observation area 1 can be displayed on the monitor 70.

Further, because the judging means 309 judges that the pixel corresponding to a target subject 2, of which the two-dimensional distribution point formed by the normalized fluorescence computed value and the computed fluorescence yield rate obtained thereof is not included in the computed value distribution data obtained of tissues of which the tissue state was known, represents an unclean tissue, the reliability of the judgment result can be improved. Further, the judgment of whether the target subject 2 is an unclean tissue can be performed concurrently with the judgment as to whether a clean tissue is a normal, precancerous or cancerous tissue, and a fluorescence diagnostic image corresponding to these results displayed on the monitor 70, whereby the diagnostician can easily discriminate between unclean, normal, precancerous and cancerous tissues, leading to an improvement in the reliability of the diagnosis.

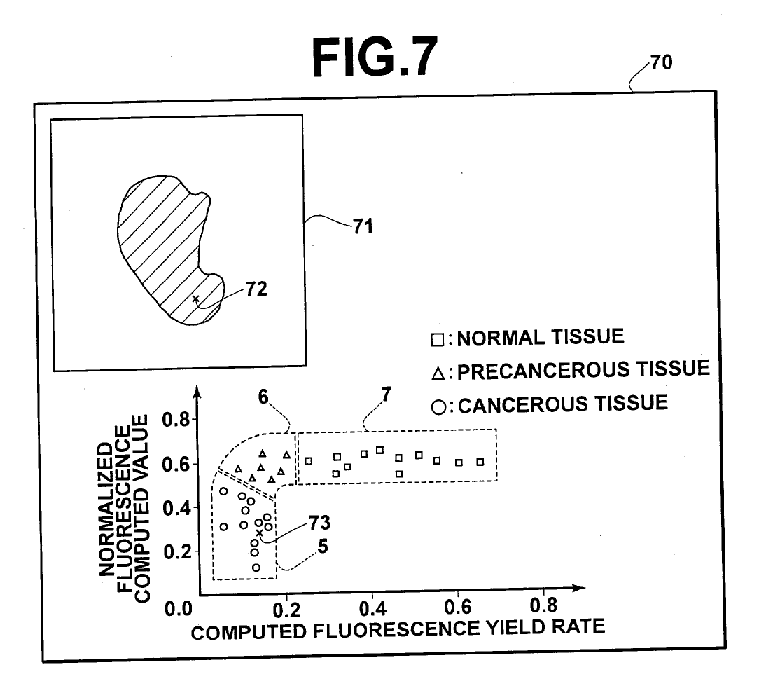

Still further, as shown in FIG. 7, the fluorescence diagnostic image 71, the two-dimensional distribution graph shown in FIG. 6 and the computed value distribution data can be displayed concurrently on the monitor 70. Because the diagnostician can observe the fluorescence diagnostic image 71, the two-dimensional distribution graph and the computed value distribution data all on one screen, the utility of the fluorescence judging apparatus is improved. Further, by adopting a configuration wherein a desired portion 72, for example, on the fluorescence diagnostic image 71 is specified by use of the input apparatus 601 to display on the two-dimensional graph a two-dimensional distribution point 73 of the region 72, visual confirmation of the tissue state of the desired position 72 is made easier. Note that in this type of case, if the display color of the two-dimensional distribution point 73 is a different color than the display colors of the already existing distribution points, the visual confirmation can be made even easier.

Note that because the above-described judgment is performed on the basis of the normalized fluorescence computed value adequately corresponding to the spectral form and a computed fluorescence yield rate corresponding to the fluorescence yield rate of the fluorescence emitted from the target subject 2, the judgment can be performed more accurately. Further, because a value obtained by dividing the intensity of the narrow band fluorescence image by the intensity of the wide band fluorescence image is used as the normalized fluorescence computed value, the possibility that a division by zero will by performed during the calculation is low, and a normalized fluorescence computed value adequately corresponding to the spectral form of the fluorescence emitted from the target subject 2 can be used.

Further, according to the current embodiment, although a judgment as to whether a target subject is a normal, precancerous or cancerous tissue has been performed, the present invention is not limited thereto; as to a variation on the current embodiment, by obtaining in advance a computed value distribution data, the judgment can be performed for patients with respect to conditions such as inflammation or edema and the like. Further, the accuracy of the judgment can be improved by recording a plurality of computed value distribution data corresponding to the medical condition to be judged, the portion of which the measurement is to be taken, and the age of the patient to be diagnosed, and appropriately switching therebetween.

Note that another possible variation on the current embodiment can be conceived of wherein the fluorescence diagnostic image forming means forms a fluorescence diagnostic image signal by first assigning green for the pixels that have been judged to represent normal tissue, yellow to pixels that have been judged to represent precancerous tissues, red to pixels that have been judged to represent cancerous tissues and no color to the pixels judged to represent unclean tissue, and then assigns no color to all of the pixel values of a pixel unit, which is formed of a predetermined number of pixels, for cases in which the ratio of the number of pixels of the pixel unit that are judged to represent unclean tissue is greater than or equal to a predetermined value.

In addition, the ratio (percentage of the display) of pixels judged to represent unclean tissue of the total number of pixels forming the fluorescence diagnostic image can be appended thereto as a reliability data, the fluorescence diagnostic image and the reliability data outputted to the video signal conversion circuit 405, converted to video signals and outputted to the monitor 70. Wherein, when the fluorescence diagnostic image is displayed on the monitor 70, the reliability data can also be displayed on the display as a percentage of the display. Note that in this case, the image itself functions as the pixel unit of the invention.

In this fashion, if the display region of the monitor corresponding to a pixel unit of which the ratio of the number of pixels that have been judged to represent unclean tissue is greater than or equal to a predetermined value is displayed colorless, it becomes easy for the diagnostician to discriminate display regions in which there are a large number of unclean tissues. Further, by displaying on the fluorescence diagnostic image, as a percentage, the ratio of the number of pixels of the total number of pixels for the fluorescence diagnostic image to be judged as representing an unclean tissue, the degree of reliability of the image can be made known to the diagnostician.

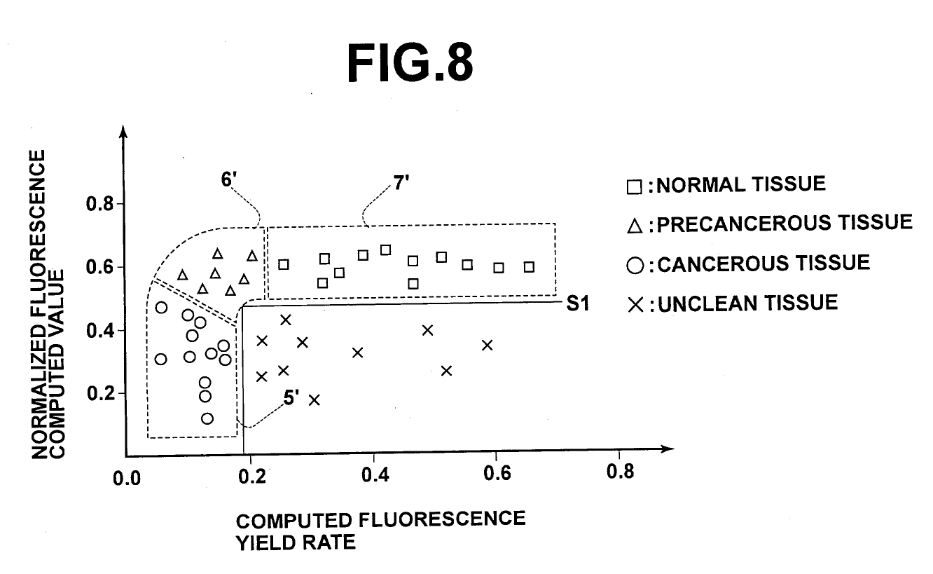

Next, the second embodiment of the present invention will be explained with reference to FIGS. 3 and 8. Because the fluorescence endoscope apparatus according to the second embodiment is substantially the same as that of the first embodiment shown in FIG. 3, reference numerals are shown only in FIG. 3. FIG. 8 illustrates an example of the computed value distribution data utilized in the current embodiment.

The fluorescence endoscope apparatus according to the current embodiment comprises, instead of the fluorescence image processing unit 30, a fluorescence image processing unit 31 provided with a signal processing circuit 301, an AD conversion circuit 302, an image memory 303, a signal processing circuit 304, an AD conversion circuit 305, an image memory 306, a fluorescence computed value obtaining means 307, a recording portion 318 for recording the computed value distribution data shown in FIG. 8, a judging portion 319 for judging, by use of the normalized fluorescence computed value and the computed fluorescence yield rate obtained by the fluorescence computed value obtaining means 307 and the computed value distribution value recorded in the memory means 318, the tissue state for each pixel; and a fluorescence diagnostic image forming means 310 for assigning a color to each pixel on the basis of the judgment result to form a fluorescence diagnostic image.

Here, the method of forming the computed value distribution data shown in FIG. 8 is explained. First, the computed fluorescence yield rate and the normalized fluorescence computed value are obtained according to the above-described method using the fluorescence endoscope apparatus of the current embodiment on the basis of the fluorescence emitted from clean tissues, to which a large quantity of fluorescence emitting mucous, digestive fluids, saliva, foam, and waste material or the like is not adhered, and from unclean tissues, to which a large quantity of mucous, digestive fluids, saliva, foam, and waste material or the like is adhered and of which the tissue state has been determined in advance by another means to be a normal state, a precancerous state, or a diseased (cancerous) state, to form a two-dimensional distribution graph relating the obtained computed values to the normal, precancerous, cancerous, and unclean tissues. Next, a threshold value S1 of a normalized fluorescence computed value and a threshold value S2 of a computed fluorescence yield rate capable of delimiting an unclean tissue area within the two-dimensional distribution graph are set. Further, the areas on the graph are delimited as an area 5′ related to the cancerous tissue, an area 6′ related to the precancerous tissue, and an area 7′ related to the normal tissue. The threshold value S1 of the normalized fluorescence computed value, the threshold value S2 of the computed fluorescence yield rate, and the range of each of the area 5′ related to the cancerous tissue, an area 6′ related to the precancerous tissue, and an area 7′ related to the normal tissue are recorded in the memory means 318 as a computed value distribution data.

The judging portion 319 judges whether or not the two-dimensional distribution point of the normalized fluorescence computed value and the computed fluorescence yield rate for each pixel falls within the range delimited by the threshold values S1 and S2. More specifically, if the normalized fluorescence computed value of a target subject 2 is less than or equal to the threshold value S1 and the computed fluorescence yield rate thereof greater than or equal to the threshold value S2, the pixel corresponding to the target subject 2 is judged to represent an unclean tissue. If the two-dimensional distribution point of a pixel falls within the normal region 7′ of the computed value distribution data shown in FIG. 8, the target subject 2 corresponding to said pixel is judged to be a normal tissue. If the distribution point of a pixel falls within the precancerous region 6′, the target subject 2 corresponding to said pixel is judged to be a precancerous tissue. If the distribution point of a pixel falls within the diseased cancerous region 5′, the target subject corresponding to said pixel is judged to be a diseased (cancerous) tissue.

The fluorescence diagnostic image forming portion 310 forms, in the same manner as occurred in the first embodiment, a fluorescence diagnostic image signal based on the judgment results, and outputs the fluorescence diagnostic image signal to the video signal processing circuit 405. The video conversion circuit 405 converts the fluorescence diagnostic image signal to a video signal, and outputs the video signal to the monitor to display the fluorescence diagnostic image.

As made clear in the above explanation, according to the fluorescence endoscope apparatus of the current embodiment, in the same manner as the first embodiment, because the tissue state of each target subject 2 of the observation area 1 is judged based on the two-dimensional distribution point formed by the normalized fluorescence computed value and the computed fluorescence yield rate of each said target subject and the computed value distribution data that has been prerecorded in the memory means 318, the accuracy of the tissue state judgment result can be improved. Further, it is also possible to judge whether the tissues state of a clean tissue is normal, precancerous or cancerous. Note that otherwise, the same result obtained by the first embodiment can also be obtained by the second embodiment.

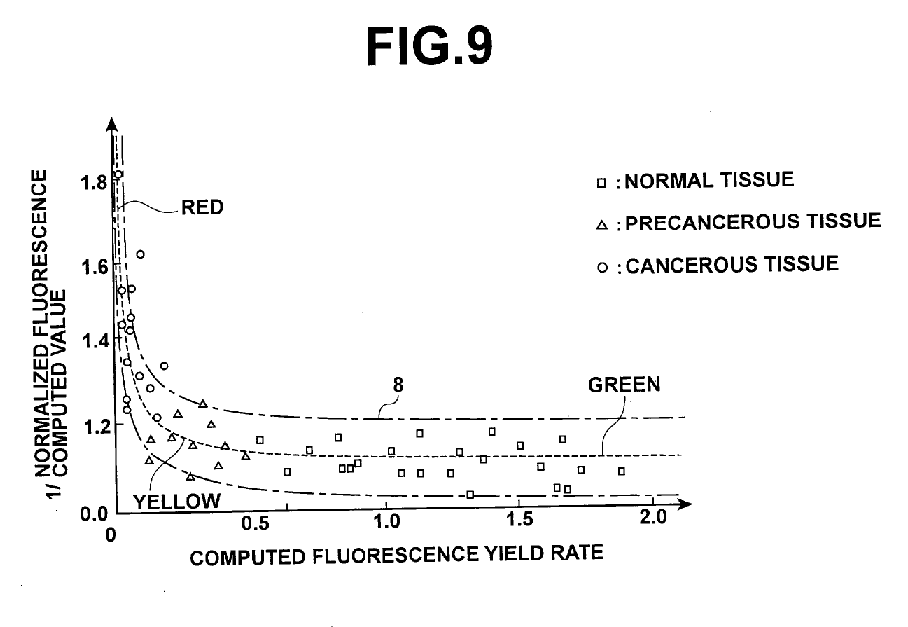

Next, the third embodiment of the present invention will be explained with reference to FIGS. 3 and 9. Because the fluorescence endoscope apparatus according to the third embodiment is substantially the same as that of the first embodiment shown in FIG. 3, the labels are shown only in FIG. 3. FIG. 9 illustrates an example of the computed value distribution data utilized in the current embodiment.

The fluorescence endoscope apparatus according to the current embodiment comprises, instead of the fluorescence image processing unit 30, a fluorescence image processing unit 32 provided with a signal processing circuit 301, an AD conversion circuit 302, an image memory 303, a signal processing circuit 304, an AD conversion circuit 305, an image memory 306, a fluorescence computed value obtaining means 307, a recording portion 328 for recording the computed value distribution data shown in FIG. 9, a judging portion 329 for judging, by use of the normalized fluorescence computed value and the computed fluorescence yield rate obtained by the fluorescence computed value obtaining means 307 and the computed value distribution value recorded in the memory means 328, the tissue state for each pixel; and a fluorescence diagnostic image forming means 330 for assigning a color to each pixel on the basis of the judgment result to form a fluorescence diagnostic image.

Here, the method of forming the computed value distribution data shown in FIG. 9 is explained. First, the computed fluorescence yield rate and the normalized fluorescence computed value are obtained according to the above-described method using the fluorescence endoscope apparatus of the current embodiment on the basis of the fluorescence emitted from clean tissues, to which a large quantity of fluorescence emitting mucous, digestive fluids, saliva, foam, and waste material or the like is not adhered, of which the tissue state has been determined in advance by another means to be a normal tissue state, a precancerous tissue state, or a diseased (cancerous) tissue state to form a two-dimensional distribution graph relating the obtained computed values to the normal, precancerous, and cancerous tissues.

Next, a computed value distribution function such as that shown by the dotted line in FIG. 9 is calculated from the two-dimension distribution graph. The computed value distribution can be represented by the following formula wherein the normalized fluorescence computed value is NF and the computed fluorescence yield rate AF.

1/NF=1.1+0.0012/AF

The standard deviation σ of the measurement value obtained from the tissues of which the tissue state is known is calculated at the same time. Further, using the standard deviation σ of the measurement value obtained from the tissues of which the tissue state is known, a clean tissue range 8 can be delimited by the following formula.

1/NF=(1.1±σ)+0.0012/AF

Note that the colors between the green (normal tissue) and yellow (precancerous tissue) ranges, and the yellow to red (cancerous tissue) ranges for each point on the computed value distribution function are set as continuous gradients. The computed value distribution function to which the colors are set and the clean tissue range 8 are recorded in the memory means 328 as the computed value distribution data.

The judging portion 329 first judges for each pixel, based on the two-dimensional distribution point of the normalized fluorescence intensity value and the computed fluorescence value, that the target subject 2 corresponding to a pixel whose two-dimensional distribution point falls outside of the clean tissue range 8 is an unclean tissue.

If the two-dimensional distribution point is within the clean tissue range 8, the point on the computed value distribution function located closest to the two-dimensional distribution point is calculated and set as the tissue state judgment point.

The fluorescence diagnostic image forming portion 330 assigns to each pixel for which the two-dimensional distribution point of the normalized fluorescence computed value and the computed fluorescence yield value thereof falls within the clean tissue range 8 the color corresponding to the tissue state judgment point on the computed value distribution function and assigns no color to the pixels that have been judged as representing unclean tissues to form a fluorescence diagnostic image signal, and outputs the fluorescence diagnostic image signal to the video signal processing circuit 405. The video conversion circuit 405 converts the fluorescence diagnostic image signal to a video signal, and outputs the video signal to the monitor 70 to display the fluorescence diagnostic image.

As made clear in the above explanation, according to the fluorescence endoscope apparatus of the current embodiment, it can be judged, based on the computed fluorescence yield rate and the normalized fluorescence computed value obtained from the observation area 1 as well as the computed value distribution data (the computed value distribution function and the clean tissue range 8) that has been recorded in advance by the memory means 328, whether the target subject corresponding to each pixel is a is a normal, precancerous or cancerous tissues; whereby, the judgment accuracy of the tissue state of the target subject 2 can be improved. Further, because the change in the tissue state within the observation area can be represented as a continuous, graduated color change, a fluorescence diagnostic image more accurately corresponding to the tissue state of the target subject 2 can be displayed on the monitor.

Note that the image regions corresponding to unclean tissues can be displayed in the same green color as the normal tissues. In this case, because the precancerous tissues are displayed as yellow, the cancerous tissues as red, and the other regions as green, the diagnostician can easily discriminate the precancerous and cancerous tissues.

According to the current embodiment also, although a judgment as to whether a target subject is a normal, precancerous or cancerous tissue has been performed, the present invention is not limited thereto; as to a variation on the current embodiment, by obtaining in advance a computed value distribution data, the judgment can be performed for patients with respect to conditions such as inflammation or edema and the like. Further, the accuracy of the judgment can be improved by recording a plurality of computed value distribution data corresponding to the medical condition to be judged, the portion of which the measurement is to be taken, and the age of the patient to be diagnosed, and appropriately switching therebetween.

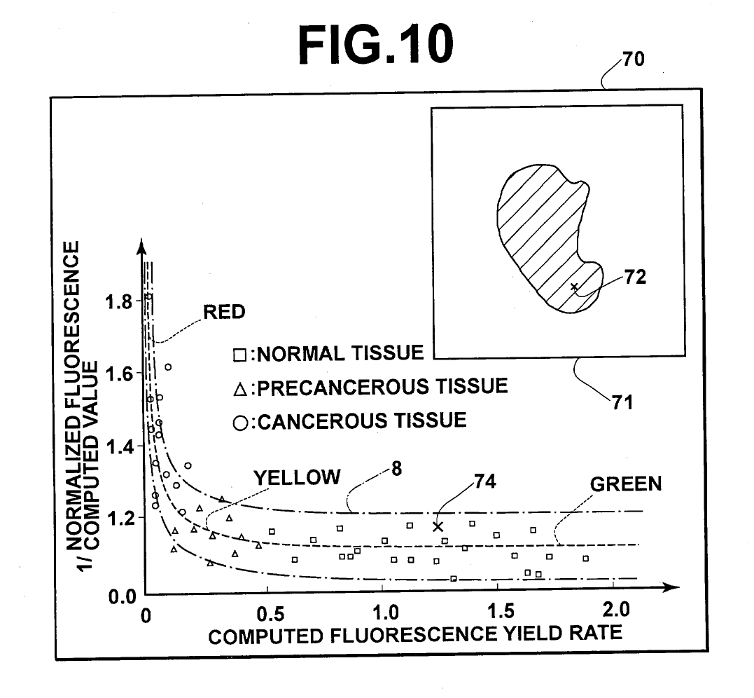

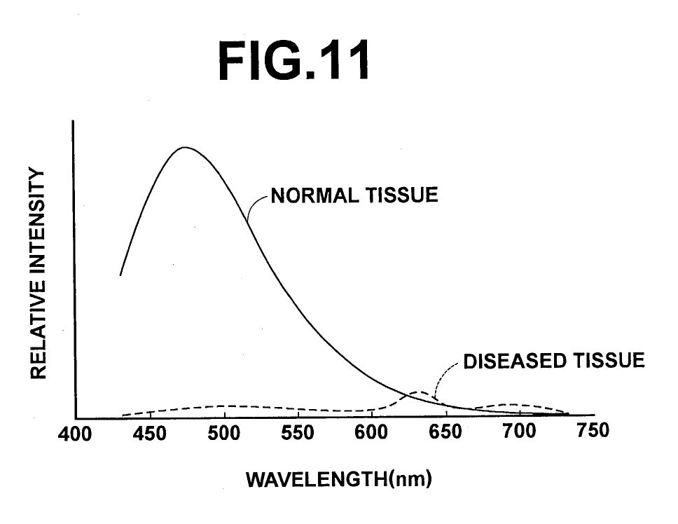

Further, as shown in FIG. 10, the fluorescence diagnostic image 71, the two-dimensional distribution graph shown in FIG. 9 and the computed value distribution data can be displayed concurrently on the monitor 70. Because the diagnostician can observe the fluorescence diagnostic image 71, the two-dimensional distribution graph and the computed value distribution data all on one screen, the utility of the fluorescence judging apparatus is improved. Further, by adopting a configuration wherein a desired portion 72, for example, on the fluorescence diagnostic image 71 is specified by use of the input apparatus 601 to display on the two-dimensional graph a two-dimensional distribution point 74 of the region 72, visual confirmation of the tissue state of the desired position 72 is made easier. Note that in this type of case, if the display color of the two-dimensional distribution point 74 is a different color than the display colors of the already existing distribution points, the visual confirmation can be made even easier. Further, when the image is to be displayed, if the deviation of the two-dimensional distribution point 74 is calculated and also displayed at the same time, the tissue state of the portion 72 can be displayed one level more accurately.

Note that as a variation on the current embodiment, it is possible that the same result as that described above can be obtained by recording the computed value distribution function and the standard deviation σ as the computed value distribution data in the memory means 328 and having the judging portion 329 calculate the clean tissue range 8 from the computed value distribution function and the standard deviation σ. Further, even for cases in which, as described above, a plurality of computed value distribution data is recorded in correspondence with the medical condition to be judged, the portion of which the measurement is to be taken, and the age of the patient to be diagnosed, and switched conveniently, because the computed value distribution function and the standard deviation a can be recorded as each computed value distribution data, the need to prepare memory for the recording of other types of tables is unnecessary.

Further, according to each embodiment, although a computed value distribution data formed based on the two-dimensional distribution graph of the normalized fluorescence intensity computation value and the computed fluorescence yield rate obtained from tissues of which the tissue state has been clearly determined in advance by another means has been used as the computed value distribution data, the present invention is not limited thereto. For example, for a case in which the tissue state, the computed fluorescence yield rate and the normalized fluorescence intensity value of a predetermined portion has been made clear by means of a biopsy performed during an endoscopy, this data can be added to recreate the computed value distribution data, and during the following endoscopy, this recreated computed value distribution data can be used to perform the tissue state judgment. Further, in accordance with the objective of the endoscopy, a configuration can be adopted wherein it is possible for the diagnostician to manually change the settings range of the computed value distribution data. For example, when a screening or the like is performed, if the computed value distribution data is set so that the range for detecting diseased tissue is wider than usual, the accuracy of the screening can be improved.

Note that according to each embodiment, although the standard images, the fluorescence images and the IR reflectance images have been obtained using one imaging element, a separate imaging element can be used for each type of image. In this case, it is desirable that each imaging element be provided with an optical filter appropriate to the wavelength range of the light to be transmitted to obtain the respective type of image. Further, a CCD imaging element can be provided in the processing portion 80, and the fluorescence image can be guided from the distal end of the scope portion 10 to the CCD imaging element within the processing portion 80.

Further, according to each embodiment, although a judgment is performed as to whether a target subject is an unclean tissues, a normal tissue, a precancerous tissue, or a cancerous tissue by use of a computed value distribution data, it is also possible to judge only whether a tissue is an unclean tissue utilizing the computed value distribution data, and to judge whether a tissue is a normal tissue, a precancerous tissue, or a cancerous tissue, that is, to judge the tissue state, by use of another means. More specifically, there are judgment methods employing only one of either the normalized fluorescence computed value or the computed fluorescence yield rate, or the like.

What is claimed is:

1. A fluorescence judging method comprising the steps of:
recording in advance a two-dimensional distribution of the fluorescence emitted from a plurality of tissues, each of which the respective tissue state is known, upon the irradiation thereof by an excitation light, wherein the two-dimensional distribution is formed of a normalized fluorescence computed value corresponding to the spectral form of the fluorescence emitted from each tissue of which the tissue state is known and a computed fluorescence yield rate corresponding to the fluorescence yield rate of the fluorescence, and a computed value distribution data formed based on the relation of the two-dimensional distribution to the tissue state of each of the tissues of which the tissue state is known,
detecting the fluorescence data of the fluorescence emitted from a target subject upon the irradiation thereof with an excitation light,
obtaining a normalized fluorescence computed value and a fluorescence yield rate computed value based on the detected fluorescence data, and
judging the tissue state of the target subject based on both of the computed values and the prerecorded computed value distribution data.

2. A fluorescence judging apparatus comprising:
a memory means for recording in advance a two-dimensional distribution of the fluorescence emitted from a plurality of tissues, each of which the respective tissue state is known, upon the irradiation thereof by an excitation light, wherein the two-dimensional distribution is formed of a normalized fluorescence computed value corresponding to the spectral form of the fluorescence emitted from each tissue of which the tissue state is known, and a fluorescence yield rate computed value corresponding to the fluorescence yield rate of the fluorescence, and a computed value distribution data formed based on the relation of the two-dimensional distribution to the tissue state of each of the respective known tissue states,
an excitation light emitting means for projecting an excitation light onto a target subject,
a fluorescence detecting means for detecting fluorescence data of the fluorescence emitted from a target subject upon the irradiation thereof with an excitation light,
a computing means for obtaining a normalized fluorescence computed value and a fluorescence yield rate computed value based on the detected fluorescence data, and
a judging means for judging the tissue state of the target subject based on both of the computed values and the prerecorded computed value distribution data.

3. A fluorescence judging apparatus as defined in claim 2, wherein
the normalized fluorescence computed value can be a value obtained by dividing the intensity of the fluorescence of the narrow wavelength range by the intensity of the fluorescence of the wide wavelength range.

4. A fluorescence judging apparatus as defined in claim 2, wherein
if the two-dimensional distribution point of the normalized fluorescence computed value and the computed fluorescence yield rate of the fluorescence emitted from the target subject is not included in the computed value distribution data, the judging means is judges that the target subject is an unclean tissue.

5. A fluorescence judging apparatus as defined in claim 3, wherein
if the two-dimensional distribution point of the normalized fluorescence computed value and the computed fluorescence yield rate of the fluorescence emitted from the target subject is not included in the computed value distribution data, the judging means is judges that the target subject is an unclean tissue.

6. A fluorescence judging apparatus as defined in claim 2, further comprising
a display means for simultaneously displaying the computed value distribution data and the judgment result of the judging means.

7. A fluorescence judging apparatus as defined in claim 3, further comprising
a display means for simultaneously displaying the computed value distribution data and the judgment result of the judging means.

8. A fluorescence judging apparatus as defined in claim 4, further comprising
a display means for simultaneously displaying the computed value distribution data and the judgment result of the judging means.

9. A fluorescence judging apparatus as defined in claim 5, further comprising
a display means for simultaneously displaying the computed value distribution data and the judgment result of the judging means.

10. A fluorescence judging method for judging the tissue state of a target subject based on fluorescence emitted from the target subject upon the irradiation thereof with an excitation light, comprising the steps of:
recording in advance distribution data of a plurality of characteristic quantities obtained based on fluorescence data of the fluorescence emitted from a clean tissue upon the irradiation thereof by an excitation light,
detecting fluorescence data of the fluorescence emitted from the target subject upon the irradiation thereof by the excitation light,
obtaining, based on the fluorescence data detected by the detecting means, the plurality of characteristic quantities, and
judging, based on the plurality of characteristic quantities and the distribution data, that the target subject is an unclean tissue.

11. A fluorescence judging apparatus for judging the tissue state of a target subject based on fluorescence emitted from the target subject upon the irradiation thereof with an excitation light, comprising:
a memory means for recording in advance distribution data of a plurality of characteristic quantities obtained based on fluorescence data of fluorescence emitted from a clean tissue upon the irradiation thereof by an excitation light,
a detecting means for detecting fluorescence data of the fluorescence emitted from the target subject upon the irradiation thereof by the excitation light,
a characteristic quantity obtaining means for obtaining, based on the fluorescence data detected by the detecting means, the plurality of characteristic quantities, and
a judging means for judging, based on the plurality of characteristic quantities and the distribution data, that the target subject is an unclean tissue.

12. A fluorescence judging apparatus as defined in claim 11, wherein
the plurality of characteristic quantities are the normalized fluorescence computed value corresponding to the spectral form of the fluorescence and a computed fluorescence yield rate corresponding to the fluorescence yield rate of the fluorescence.

13. A fluorescence judging apparatus as defined in claim 11, wherein
the clean tissue is composed of a plurality of tissues of which the tissue state is known,
the memory means is a means for recording in advance the distribution data of a plurality of characteristic quantities for each of the tissues of which the tissue state is known,
the judging means is a means for judging the tissue state of the target subject based on the plurality of characteristic quantities obtained by the characteristic quantities obtaining means and the distribution data of the characteristic quantities for each of the tissues of which the tissue state is known.

14. A fluorescence judging apparatus as defined in claim 12, wherein
the clean tissue is composed of a plurality of tissues of which the tissue state is known,
the memory means is a means for recording in advance the distribution data of a plurality of characteristic quantities for each of the tissues of which the tissue state is known,
the judging means is a means for judging the tissue state of the target subject based on the plurality of characteristic quantities obtained by the characteristic quantities obtaining means and the distribution data of the characteristic quantities for each of the tissues of which the tissue state is known.

15. A fluorescence judging apparatus as defined in claim 13, wherein
the excitation light emitting means projects excitation light onto an observation area,
the detecting means detects as an image fluorescence data of fluorescence emitted from the observation area,
the characteristic quantities obtaining means obtains, based on the fluorescence data, a plurality of characteristic quantities for each pixel of the image, and
the judging means judges, based on the plurality of characteristic quantities and the distribution data that has been prerecorded in the memory means, for each pixel that the target subject corresponding to the pixel is an unclean tissue, further comprising
a fluorescence diagnostic image forming means for forming a fluorescence diagnostic image based on the judgment results obtained by the judging means, and
a display means for displaying the fluorescence diagnostic image.

16. A fluorescence judging apparatus as defined in claim 14, wherein
the excitation light emitting means projects excitation light onto an observation area,
the detecting means detects as an image fluorescence data of the fluorescence emitted from the observation area,
the characteristic quantities obtaining means obtains, based on the fluorescence data, a plurality of characteristic quantities for each pixel of the image, and
the judging means judges, based on the plurality of characteristic quantities and the distribution data that has been prerecorded in the memory means, for each pixel that the target subject corresponding to the pixel is an unclean tissue, further comprising
a fluorescence diagnostic image forming means for forming a fluorescence diagnostic image based on the judgment results obtained by the judging means, and
a display means for displaying the fluorescence diagnostic image.

17. A fluorescence judging apparatus as defined in claim 15, wherein
the fluorescence diagnostic image forming means is a means for subjecting a pixel unit, which is formed of a predetermined number of pixels, to a display correction process for cases in which the ratio of the number of pixels of a pixel unit that are judged to represent unclean tissue is greater than or equal to a predetermined value.

18. A fluorescence judging apparatus as defined in claim 16, wherein
the fluorescence diagnostic image forming means is a means for subjecting a pixel unit, which is formed of a predetermined number of pixels, to a display correction process for cases in which the ratio of the number of pixels of a pixel unit that are judged to represent unclean tissue is greater than or equal to a predetermined value.

19. A fluorescence judging apparatus as defined in claim 15, wherein
the fluorescence diagnostic image forming means is a means for appending reliability data, corresponding to the ratio of the number of pixels judged to represent images of unclean tissue of a pixel unit, which is formed of a predetermined number of pixels, to the pixel unit.

20. A fluorescence judging apparatus as defined in claim 16, wherein
the fluorescence diagnostic image forming means is a means for appending a reliability data, corresponding to the ratio of the number of pixels judged to represent images of unclean tissue of a pixel unit, which is formed of a predetermined number of pixels, to the pixel unit.

21. A fluorescence judging apparatus as defined in claim 17, wherein
the fluorescence diagnostic image forming means is a means for appending a reliability data, corresponding to the ratio of the number of pixels judged to represent images of unclean tissue of a pixel unit, which is formed of a predetermined number of pixels, to the pixel unit.

22. A fluorescence judging apparatus as defined in claim 18, wherein
the fluorescence diagnostic image forming means is a means for appending a reliability data, corresponding to the ratio of the number of pixels judged to represent images of unclean tissue of a pixel unit, which is formed of a predetermined number of pixels, to the pixel unit.
